(19) United States
(12) Patent Application Publication
(10) Pub. No.: US 2004/0038932 A1
(43) Pub. Date: Feb. 26, 2004

(54) Antithrombotic compositions

(76) Inventors: Jack Hirsh, Hamilton (CA); Kristian Johansen, Haellebaek (DK); Jeffrey I. Weitz, Ancaster (CA)

Correspondence Address: MERCHANT & GOULD PC, P.O. BOX 2903, MINNEAPOLIS, MN, 55402-0903, US

(21) Appl. No.: 10/363,957

(22) PCT Filed: Sep. 7, 2001
(86) PCT No.: PCT/CA01/01287

(30) Foreign Application Priority Data
Sep. 8, 2000 (US) 60231429

(51) Int. Cl. A61K 31/727; A61K 31/737

(52) U.S. Cl. 514/54; 514/56

(57) ABSTRACT

The invention relates generally to compositions and methods for preventing or inhibiting thrombin generation or activity.

### FIELD OF THE INVENTION

The invention relates generally to compositions and methods for inhibiting or preventing thrombin generation or activity.

### BACKGROUND OF THE INVENTION

Excessive generation of thrombin, which is characteristic of diseases including heart attack, stroke and deep vein thrombosis, can be life threatening and requires effective treatment There are various commercial preparations that block the effects of thrombin. Heparin, a sulfated polysaccharide, acts as an anticoagulant by accelerating the inhibition of thrombin and factor Xa by antithrombin (AT) (1). Although heparin is widely used for the treatment of acute coronary ischemic syndromes, it has limitations in patients undergoing percutaneous coronary interventions (2), or when used as an adjunct to thrombolytic therapy (3). These limitations have been attributed to the inability of the AT-heparin complex to inactivate clotting enzymes bound to components of the thrombus, particularly thrombin bound to fibrin (4,5).

Dermatan sulfate (DS), a sulfated glycosaminoglycan that has antithrombotic activity in laboratory animals (6-9) and in humans (10-13), acts as an anticoagulant by catalyzing only heparin cofactor II (HCII). Since thrombin is the exclusive plasma target of HCII, DS is considered to be a selective inhibitor of thrombin (14). Although fibrin-bound thrombin is protected from inactivation by the heparin-HCII complex (15), indirect studies done in plasma systems suggest that fibrin-bound thrombin is susceptible to inactivation by the DS-HCII complex (16). However, dermatan sulfate has practical clinical limitations because of its low specific biological activity combined with high viscosity.

The addition of dermatan sulfate to a commercially available low molecular weight heparin (either Enoxaparin or Fragmin) had an additive effect on thrombin inhibition in vitro (17). However, the tested combination is not clinically useful because it is not effective against fibrin-bound thrombin, and the DS has low potency and poor solubility.

### SUMMARY OF THE INVENTION

It has been demonstrated that the combination of an oligosaccharide fraction obtained from heparin (herein also referred to as “heparin oligosaccharide fraction”) and an oligosaccharide fraction obtained from dermatan sulfate (herein also referred to as “dermatan sulfate oligosaccharide fraction) provides advantageous inhibitory effects on both fluid-phase and fibrin-bound thrombin. Selected combinations of the heparin oligosaccharide fraction and dermatan oligosaccharide fraction provided unexpectedly greater than additive i.e. synergistic inhibitory effects. The new concept of the invention is to combine the oligosaccharide fractions to ensure maximum anticoagulant activity of heparin and dermatan sulfate without increasing the risk of bleeding.

Each ollgosaccharide fraction in the combination therapy is expected to inhibit thrombin by a different mechanism. While not wishing to be bound by theoretical mechanisms of action, the heparin oligosaccharide fraction can inhibit fibrin-bound thrombin as well as fluid-phase thrombin by activating antithrombin, and it can inhibit thrombin generation by catalyzing factor Xa inactivation by antithrombin. The dermatan sulfate oligosaccharide fraction can inhibit fibrin-bound thrombin by activating HCII. In its activated conformation, the amino-terminal domain of HCII binds to exosite I. Because thrombin binds to fibrin via exosite I, activated HCII competes with fibrin for thrombin binding and displaces thrombin from fibrin. Displaced thrombin can then be inactivated by heparin/HCII, dermatan sulfate/HCII, or heparin/antithrombin.

A combination of therapies that inhibit thrombin by different mechanisms to achieve maximum efficacy, will improve tolerance to the therapy, and result in a reduced risk of side effects that can be caused by high-dose and long-term use of the drugs in monotherapy. Therefore, a combination treatment of the invention will permit the use of lower doses of each component (e.g. at least 5 to 10 fold lower doses as illustrated in Example 2), with reduced adverse, toxic effects of each component A lower dosage may provide an increased margin of safety relative to the margin of safety for each component when used as a single agent. In addition, where a convenient single combination dosage unit is offered to a patient, it is generally accepted that the increased convenience will result in an increase in compliance, as well as reducing the likelihood of patient confusion often associated with multiple dosage unit forms of medications.

Broadly stated, the present invention relates to a combination treatment for inhibiting or preventing thrombin generation or activation in a patient comprising administering to the patient an effective amount of (a) at least one heparin oligosaccharide fraction; and (b) at least one dermatan sulfate oligiosaccharide fraction. In an aspect of the invention the combination treatment provides synergistic activity. In another aspect a method of inhibiting or preventing thrombin generation or activity in a patient is provided comprising administering in combination, to a patient in need thereof, effective amounts (preferably synergistically effective amounts) of at least one heparin oligosaccharide fraction, and at least one dermatan sulfate oligosaccharide fraction.

“Combination treatment” or “administering in combination” means that the active ingredients are administered concurrently to a patient being treated When administered in combination, each component may be administered at the same time or sequentially in any order, and at different points in time. Therefore, each component may be administered separately, but sufficiently close in time to provide the desired effect (preferably a synergistic effect).

The present invention also provides compositions comprising a combination of (a) at least one heparin oligosaccharide fraction; and (b) at least one dermatan sulfate oligosaccharide fraction, optionally together with a pharmaceutically acceptable excipient, carrier, or vehicle. The present invention also contemplates a pharmaceutical composition in separate containers and intended for simultaneous or sequential administration, comprising a heparin oligosaccharide fraction, and a dermatan sulfate oligosaccharide fraction, both optionally together with pharmaceutically acceptable excipients, carriers, or vehicles.

In another embodiment, the invention provides a pharmaceutical composition comprising a unit dosage of at least one heparin oligosaccharide fraction; and a unit dosage of at least one dermatan sulfate oligosaccharide fraction, optionally together with a pharmaceutically acceptable excipient, carrier, or vehicle.

The above mentioned compositions also include pharmaceutically acceptable salts of the heparin oligosaccharide and dermatan sulfate oligosaccharide fractions, such as sodium, potassium, ammonia, magnesium, and calcium salts.

In accordance with one aspect, a pharmaceutical composition is provided comprising a combination of (a) at least one heparin oligosaccharide fraction; and (b) at least one dermatan sulfate oligosaccharide fraction effective to exert a synergistic effect in preventing or inhibiting thrombin generation or activity. The method also provides pharmaceutical compositions comprising a synergistically effective amount of a combination of at least one heparin oligosaccharide fraction and at least one dermatan sulfate oligosaccharide fraction, in A pharmaceutically acceptable excipient, carrier, or vehicle.

In a preferred embodiment, the pharmaceutical compositions comprise a heparin oligosaccharide fraction and a dermatan sulfate oligosaccharide fraction in doses that are at least 5 to 10 fold lower than the doses of each fraction required to prevent or inhibit thrombin generation or activity in a patient In another aspect the invention relates to the use of a composition comprising a combination of (a) at least one heparin oligosaccharide fraction and (b) at least one dermatan sulfate oligosaccharide fraction for the preparation of a medicament for the prevention or inhibition of thrombin generation or activity. In another aspect the invention relates to the use of synergistically effective amounts of at least one heparin oligosaccharide fraction, and at least one dermatan sulfate oligosaccharide fraction in the preparation of a pharmaceutical composition for inhibiting or preventing thrombin generation or activity in a patient.

Since the present invention relates to a method of treatment comprising a combination of active agents which may be administered separately, the invention also relates to combining separate compositions comprising the active agents in kit form.

The invention also relates to an oligosaccharide fraction of the invention, and compositions and treatments as described generally herein using such fractions.

The invention also contemplates the use of a composition of the invention or combination treatment of the invention for preventing, and/or ameliorating disease severity, disease symptoms, and/or periodicity of recurrence of a disease associated with excess thrombin generation or activity.

These and other aspects, features, and advantages of the present invention should be apparent to those skilled in the art from the following drawings and detailed description.

### DESCRIPTION OF THE DRAWINGS

The invention will be better understood with reference to the drawing in which:

FIG. 1 shows the effects of soluble fibrin (Fm) on the second-order rate constants for the inhibition of thrombin by DS-catalyzed (panel A) or heparin-catalyzed (panel B) HCII. The second-order rate constants for the DS- or heparin-catalyzed inhibition of 10 nM thrombin by 100 nM HCII in the absence or presence of Fm were determined under pseudo first-order conditions. Each point represents the mean of four determinations, and the bars represent the standard error.

FIG. 2 is a bar graph showing the effect of 4 μM fibrin monomer on antithrombin (AT) inhibition of thrombin with heparinase derived heparin oligosaccharide fractions of increasing molecular weight.

FIG. 3 shows the effect of heparin and DS on the binding of thrombin to fibrin. The binding of thrombin to fibrin clots was determined in the presence of increasing concentrations of either heparin (●) or DS (o). Each point represents the mean of two determinations and the bars represent the standard error.

FIG. 4 is a graph showing the effect of heparin or heparinase-derived fractions on thrombin binding to fibrin clots.

FIG. 5 is a graph showing the displacement of thrombin from fibrin by HCII in the presence of DS or standard heparin (SH).

FIG. 6A is a graph showing the effect of a 5:1 combination of heparin oligosaccharide fraction: dermatan sulfate oligosaccharide fraction versus heparin oligosaccharide fraction alone on cumulative patency in a rabbit arterial thrombosis prevention model.

FIG. 6B is a graph showing the effect of a 5:1 combination of heparin oligosaccharide fraction: dermatan sulfate oligosaccharide fraction versus heparin oligosaccharide fraction alone on cumulative blood loss in a rabbit arterial thrombosis model.

FIG. 7 is a graph showing a comparison of the effect of heparin, LMWH, a heparin oligosaccharide fraction, and hirudin on patency (Panel A) and a graph showing a comparison of the effects on blood loss Panel B).

FIG. 8 is a graph showing the effect of a heparin oligosaccharide fraction on platelet deposition on vascular grafts in a baboon arterial thrombosis model.

FIG. 9 is a graph showing the effect of a dermatan sulfate oligosaccharide fraction on platelet deposition on vascular grafts in a baboon arterial thrombosis model.

FIG. 10 is a graph showing the effect of a combination of a heparin oligosaccharide fraction and a dermatan sulfate oligosaccharide fraction on platelet deposition on vascular grafts in a baboon arterial thrombosis model.

### DETAILED DESCRIPTION OF THE INVENTION

Heparin Oligosaccharide Fraction

“Oligosaccharide fraction obtained from heparin” or “heparin oligosaccharide fraction” refers to a mixture of oligosaccharides derived from heparin characterized by having antithrombin- and HCII-related anticoagulant activity in vitro. The fraction comprises heparin chains that are too short to bridge thrombin to fibrin, but are of a sufficient length to bridge antithrombin or HCII to thrombin.

The fraction may comprise a mixture of oligosaccharides derived from heparin characterized by one, two, three, four, five, six, or seven or more of the following characteristics:

(a) having antithrombin- and heparin cofactor II (HCII) related anticoagulant activity in vitro;

(b) the oligosaccharides are too short to bridge thrombin to fibrin, but are of a sufficient length to bridge antithrombin or HCII to thrombin;

(c) having at least 15%, 20%, 25%, 30%, 35%, or 40% oligosaccharides with at least one or more pentasaccharide sequence;

(d) enriched for oligosaccharides having a molecular weight range from about 6,000 to about 12,000, 6,000 to 11,000, or 6,000 to 10,000;

(e) the oligosaccharides have a peak molecular weight of about 7,000 to 10,000, 7,500 to 9,700, or 8,000 to 9,000;

(f) at least 30%, 35%, 40%, 45%, 50%, or 55% of the oligosaccharides have a molecular weight greater than or equal to 6000 Daltons;

(g) at least 20%, 25%, 30%, 35%, or 40%, have a molecular weight greater than or equal to 8000 Daltons;

(h) a polydispersity of 1.1 to 1.8, particularly 1.2 to 1.7; and

(i) having similar anti-factor Xa and anti-factor Ila activities, preferably a ratio of anti-factor Xa activity to anti-factor Ila activity from about 2:1 to about 1:1.

In accordance with an aspect of the invention a fraction used in the present invention has the characteristics (a), (b), (c) and (d); (a) (b), (c), and (e); (a), (b), (e), and (f); (a), (b), (e), and (g); (a), (b), (e), (f), (g), (h) and (i); (b), (c), (e), and (g); (b), (d), (c), (h), and (i); (b) (c), (d), and (h); (b), (e), (h), and (i); (b), (e), (f), (h), and (i); (b), (e), (g), (h), and (i); or (a) through (i).

“Enriched for oligosaccharides” refers to a fraction comprising at least 20%, 25%, 30%, 35%, 40%, 45%, or 50% oligosaccharides within a specified or restricted molecular weight range.

“Pentasaccharide sequence” refers to a key structural unit of heparin that consists of three D-glucosamine and two uronic acid residues (See the structure below). The central D-glucosarnine residue contains a unique 3-O-sulfate moiety.

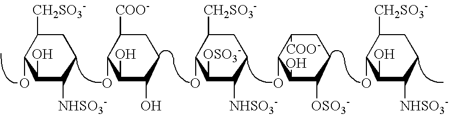

The pentasaccharide sequence represents the ninimum structure of heparin that has high affinity for antithrombin (Choay, J. et al., Biochem Biophys Res Comm 1983; 116: 492-499). The binding of heparin to antithrombin through the pentasaccharide sequence results in a conformational change in the reactive center loop which converts antithrombin from a slow to a very rapid inhibitor.

A heparin oligosaccharide fraction comprises heparin chains that are too short to bridge thrombin to fibrin, but are of sufficient length to bridge antithrombin to thrombin. Consequently, a fraction selected for use in the present invention will be capable of inhibiting fibrin-bound thrombin as well as fluid-phase thrombin by catalyzing antithrombin, and inhibiting thrombin generation by catalyzing factor Xa inactivation by antithrombin. Preferably, fractions selected for use in the present invention are those that inhibit fibrin-bound thrombin and fluid-phase thrombin equally well.

Characteristics of suitable heparin oligosaccharide fractions that may be used in the present invention are set out in Table 1 and Table 2.

A heparin oligosaccharide fraction employed in the present invention may have similar anti-factor Xa and anti-factor IIa activities. In an embodiment, the ratio of anti-factor Xa activity to anti-factor IIa activity ranges from about 2:1 to about 1:1. In a preferred embodiment, the anti-factor Xa activity ranges from about 80 IU/mg to about 155 IU/mg, preferably 90 IU/mg to about 140 IU/mg in a preferred embodiment, the anti-factor IIa activity ranges from about 20 IU/mg to about 150 IU/mg; more preferably 40 IU/mg to about 130 IU/mg.

The compositions, methods, and kits of the present invention may use the modified heparin compositions described in PCT/CA98/00548 (WO98/55515, published Dec. 10, 1998), U.S. application Ser. No. 60/141,865 filed Jun. 30, 1999, or U.S. application Ser. No. 60/154,744 filed Sep. 17, 1999, which are incorporated herein by reference.

It will be appreciated that it may be possible to produce a heparin oligosaccharide fraction for use in the present invention that has more particular characteristics that are within those set out in (d), (e), (h), and (i) above. For example, the fraction may have a molecular weight range from about 7,000 to 10,000; 7,500 to 10,000; 7,800 to 10,000; 7,800 to 9,800; 7,800 to 9,600; 7,800 to 9,000; 7,800 to 8,800; 7,800 to 8,600; 7,800 to 8,500; or 8,000 to 8,500. It may also be possible to use a heparin oligosaccharide fraction that has a peak molecular weight of 7,800 to 10,000; 7,800 to 9,800; 7,800 to 9,600; 7,800 to 9,000; 7,800 to 8,800; 7,800 to 8,600; 7,800 to 8,500; or 8,000 to 8,500. A fraction employed in the compositions and methods of the invention may be developed that has a polydispersity of 1.3 to 1.6. In addition, fractions may be developed for use in the present invention that have one or more of the following particular characteristics: (i) a ratio of anti-factor Xa activity to anti-factor IIa activity of from about 1.5:1 to about 1:1; (ii) anti-factor Xa activity of from about 95 IU/mg to about 120 IU/mg or from about 100 to 110 IU/mg; (iii) anti-factor Ha activity of from about 80 IU/mg to about 100 IU/mg or from about 90 to 100 IU/mg.

In an embodiment of the invention, the heparin oligosaccharide fraction has one or more of the following characteristics:

(a) enriched for oligosaccharides with a molecular weight range from 6,000 to 10,000 Daltons, and a mean of about 8,500;

(b) white to off-white crystalline solid;

(c) stable at room temperature; and

(d) a polydispersity of about 1.2 to 1.5, preferably 1.5.

A heparin oligosaccharide fraction for use in the present invention can be obtained from tissues in a manner conventional for the preparation of such oligosaccharides of heparin, or it can be otherwise synthesize. In particular, a heparin oligosaccharide fraction may be prepared from unfractionated heparin or, alternatively, from low molecular weight heparin (LMWH).

By way of example, a heparin oligosaccharide fraction can be obtained from unfractionated heparin by first depolymerizing the unfractionated heparin to yield a lower molecular weight heparin, and isolating or separating out a heparin oligosaccharide fraction of interest The unfractionated heparin can be either a commercial heparin preparation of pharmaceutical quality or a crude heparin preparation, such as is obtained upon extracting active heparin from mammalian tissues or organs. Commercial heparin product (USP heparin) is available from several sources (e.g., SIGMA Chemical Co., St Louis, Mo.), generally as an alkali metal or alkaline earth salt (most commonly as sodium heparin). Alternatively, the unfractionated heparin can be extracted from mammalian tissues or organs, particularly from intestinal mucosa or lung from, for example, beet, porcine and sheep, using a variety of methods known to those skilled in the art (see, e.g., Coyne, Erwin, Chemistry and Biology of Heparin, (Lundblad, R. L., et al. (Eds.), pp. 9-17, Elsevier/North-Holland, N.Y. (1981)). In a preferred embodiment, the unfractionated heparin is porcine intestinal heparin.

Many processes for the depolymerization of heparin are known, and they are generally based on either chemical or enzymatic reactions. For instance, a heparin oligosaccharide fraction of the invention can be prepared from standard, unfractionated heparin by benzylation followed by alkaline depolymerization; nitrous acid depolymerization; enzymatic depolymerization with heparinase; peroxidative depolymerization, etc. In particular, a heparin oligosaccharide fraction may be prepared using the nitrous acid depolymerization method or periodate oxidation hydrolysis method described in PCT/CA98/00548 (WO98/55515).

In an embodiment, a heparin oligosaccharide fraction is prepared from unfractionated heparin using heparinase depolymerization (see for example, U.S. Pat. No. 3,766,167, and U.S. Pat. No. 4,396,762). In a preferred embodiment a fraction is prepared by a controlled heparinase depolymerization.

Dermatan Sulfate Oligosaccharide Fraction

“Oligosaccharide fraction obtained from dermatan sulfate”, or “dermatan sulfate oligosaccharide fraction” refers to a mixture of oligosaccharides derived from dermatan sulfate characterized by having little or no antithrombin-related activity, but having HCII related anticoagulant activity in vitro. Dermatan sulphate consists of alternating uronic acid and N-acetylgalactosamine residues. Many glucuronic acid residues become epimerised at C-5 to yield iduronic acid residues. Subsequently, O-sulphation may occur at the C-4 or C-6 position of GaINAc or at the C-2 position of IdoA. The fractions employed in the present invention show higher affinity towards HCII than native unfractionated dermatan sulfate.

A dermatan sulfate oligosaccharide fraction selected for use in the present invention is characterized by one or more, preferably all of the following:

(a) a sulfur content of 6.0% to 10.0% (w/w), e.g. from 6.0 to 8.0% (w/w) , preferably 6.5% to 8% (w/w);

(b) a sulfate/carboxyl ratio of 1.2 to 2.5, e.g. from 12 to 2.0, e.g. from 1.3 to 1.8, preferably from 1.3-1.6;

(c) a disulfated disaccharide c ntent of 20% to 60% (w/w), preferably 30% to 60% (w/w) of the mono-sulfated disaccharide content;

(d) a heparin cofactor II mediated activity against thrombin in the range 20-60 IU/mg, preferably 30-60 IU/mg.

In an embodiment of the invention, a dermatan sulfate oligosaccharide fraction is selected that comprises a mixture of dermatan sulfate oligosaccharides with 90% or more having a molecular weight ranging between about 1600 to about 20,000 Daltons and a peak molecular weight from about 4,500 to about 8,000 Daltons.

In a preferred embodiment of the invention, the dermatan sulfate oligosaccharide fraction has one r more of the following characteristics:

(a) enriched for oligosaccharides with a molecular weight range from 5,000 to 8,000 Daltons;

(b) white to off-white crystaline solid;

(c) stable at room temperature; and

(d) greater than 6.2% sulfonation by weight.

A dermatan sulfate oligosaccharide fraction may be obtained from tissues in a manner conventional for the preparation of such oligosaccharides from unfractionated dermatan sulfate, or it can be otherwise synthesized de novo from the relevant monosaccharides. Preferably, a depolymerizrtion method that protects and facilitates the isolation of highly charged regions of unfractionated dermatan sulfate is used to provide fractions for use in the present invention that have improved solubility and potency compared to unfractionated dermatan sulfate. For example, a dermatan sulfate oligosaccharide fraction may be prepared by the following steps: oxidation and depolymerization of dermatan sulfate by periodate oxidation, borohydride reduction, acid hydrolysis, and ion exchange chromatography.

Sources of the dermatan sulfate that can be used to prepare the fractions include mammalian tissues, for example, mammalian skin, including vascularized tissue and skin from porcine or bovine sources. Preferably intestinal mucosa is used as a source of dermatan sulfate.

The compositions, methods, and kits of the present invention preferably use a dermatan sulfate oligosaccharide mucure, and methods for preparing such mixtures, as described in PCT/EP98/03007 (WO 98/55514 published Dec. 10, 1998), which is incorporated herein by reference.

Determination of Properties of Oligosaccharide Fractions

The molecular weight characteristics of a heparin or dermatan oligosaccharide fraction employed in the present invention can be determined using standard techniques known to and used by those of skill in the art. Such techniques include, for example, GPC-HPLC, viscosity measurements, light scattering, chemical or physical-chemical determination of functional groups created during the depolymerization process, etc. In a preferred embodiment, the molecular weight characteristics of an oligosaccharide fraction is determined by high performance size exclusion chromatography.

In particular, the following methods may be used to confirm the properties and characteristics of a heparin or dermatan oligosaccharide fraction used in the present invention:

(a) Molecular weight by GPC-HPLC according to the method of Dedem, Pharmeuropa, 3, 202-218, 1991.

(b) Sulfur content according to Ph. Eur. 2.ed. V.3.53.

(c) Sulphate/carboxylate ratio according to Ph. Eur. 1997:0828.

(d) HCII mediated antithrombin activity by a chromogenic assay (Diagnostica Stago, France) in a plasma free system with the 4. International Heparin Standard (code no. 82/502) as standard.

(e) Anti-factor Xa and antifactor IIa according to Ph. Eur. 1997:0828, both methods modified using the statistical methods for slope-ratio assays.

Compositions and Methods

The compositions and methods of the invention are useful in therapeutic applications for the prevention or treatment of conditions or diseases that are characterized by excess thrombin generation or activity, and/or excess complement activation. Such conditions often occur where a subject has been exposed to trauma, for example in surgical patients. Trauma caused by wounds or surgery results in vascular damage and secondary activation of blood coagulation. These undesirable effects may occur after general or orthopedic surgery, gynecologic surgery, heart or vascular surgery, or other surgical procedures. Excess thrombin may also complicate progression of natural diseases such as artherosclerosis which can cause heart attacks, strokes or gangrene of the limbs. Therefore, the methods and compositions of the present invention can be used to treat, prevent, or inhibit a number of important cardiovascular complications, including unstable angina, acute myocardial infarction (heart attack), cerebral vascular accidents (stroke), pulmonary embolism, deep vein thrombosis, arterial thrombosis, etc. The compositions and methods of the invention may be used to reduce or prevent clotting during dialysis and reduce or prevent intravascular coagulation during open heart surgical procedures. They may also be used to maintain the patency of medical devices such as i.v. injection devices.

In one aspect of the invention, methods and compositions are provided for preventing or inhibiting thrombin generation or activity in patients at increased risk of developing a thrombus due to medical conditions that disrupt hemostsis (e.g., coronary artery disease, atherosclerosis, etc.). In another aspect, methods and compositions are provided for patients at increased risk of developing a thrombus after a medical procedure, such as cardiac surgery, vascular surgery, or percutaneous coronary interventions. In an embodiment, the methods and compositions of this invention are used in cardiopulmonary bypass. The compositions, or oligosaccharide fractions in a method of the invention, can be administered before, during or after the medical procedure.

Therapeutic efficacy and toxicity may be determined by standard pharmaceutical procedures in cell cultures or with experimental animals such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

Patients that may receive a combination treatment or be administered a composition of the invention include animals, including mammals, and particularly humans. Animals also include domestic animals, including horses, cows, sheep, poultry, fish, pigs, cats, dogs, and zoo animals.

The compositions of the present invention, a heparin oligosaccharide fraction, or a dermatan sulfate oligosaccharide fraction, can be administered by any means that produce contact of an active agent with the agent's sites of action in the body of the patient The heparin and dermatan sulfate oligosaccharide fractions can be administered simultaneously or sequentially in any order, and at different points in time, to provide the desired effect It lies within the capability of a skilled physician or veterinarian to chose a dosing regime that optimizes the effects of the compositions and treatments of the present invention. The compositions may be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. The compositions of the invention may be administered in intranasal form via topical use of suitable intranasal vehicles, or via transdermal routes, for example using conventional transdermal skin patches. The dosage administration in a transdermal delivery system will be continuous rather than intermittent throughout the dosage regimen.

The present invention includes combination treatments providing synergistic activity or delivering synergistically effective amounts of dermatan sulfate and heparin oligosaccharide fractions. Pharmaceutical compositions suitable for use in the present invention include compositions wherein the active ingredients are contained in a synergistically effective amount. By “synergistic activity” or “synergistically effective amount” is meant that a sufficient amount of the heparin oligosaccharide fraction and dermatan sulfate oligosaccharide fraction will be present in order to achieve a desired result that is greater than the result achieved with each fraction on its own, e.g. improved inhibition of thrombin accretion when treating a thrombus-related cardiovascular condition, such as those described above by, for example, improved inactivation of clot-bound thrombin, improved inhibition of thrombin generation by catalyzing factor Xa inactivation by antithrombin, etc.

The dosage regimen of the invention will vary depending upon known factors such as the pharmacodynamic characteristics of the particular agent and its mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect The effective amount of a drug required to prevent, counter, or arrest progression of a condition can be readily determined by an ordinarily skilled physician or veterinarian.

A composition or treatment of the invention may comprise a unit dosage of at least one heparin oligosaccharide fraction and a unit dosage of at least one dermatan sulfate oligosaccharide fraction. A “unit dosage” refers to a unitary i.e. a single dose which is capable of being administered to a patient, and which may be readily handled and packed, remaining as a physically and chemically stable unit dose comprising either the active agent as such or a mixture of it with solid or liquid pharmaceutical excipients, carriers, or vehicles.

Typically, the active agents i.e., the heparin ollgosaccharide fraction and dermatan sulfate oligosaccharide fraction, will each be present in a pharmaceutical composition (or used in a treatment of the invention) at a concentration ranging from about 2 mg per dose to 1000 mg per dose and, more preferably, at a concentration ranging from about 5 mg per dose to 500 mg per dose. Daily dosages can vary widely, but will usually be present at a concentration ranging from about 20 mg per dose per day to about 100 mg per dose per day and, more preferably, at a concentration ranging from about 40 mg per dose per day to about 80 mg per dose per day.

The ratio of heparin oligosaccharide fraction to dermatan sulfate oligosaccharide fraction in a composition or treatment of the invention may be 1:1 to 10:1, preferably 1:1 to 8:1, more preferably 2:1 to 6:1, most preferably 5:1.

The compositions of the present invention or fractions thereof typically comprise suitable pharmaceutical diluents, excipients, vehicles, or carriers selected based on the intended form of administration, and consistent with conventional pharmaceutical practices. The carriers, vehicles etc. may be adapted to provide a synergistically effective amount of the active fractions to inhibit or prevent thrombin generation or activity in a patient.

Suitable pharmaceutical diluents, excipients, vehicles, and carriers are described in the standard text, Remington's Pharmaceutical Sciences, Mack Publishing Company. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof.

Formulations for parenteral administration of a composition of the invention may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

A composition of the invention may be sterilized by, for example, filtration through a bacteria retaining filter, addition of sterilizing agents to the composition, irradiation of the composition, or heating the composition. Alternatively, the fractions of the present invention may be provided as sterile solid preparations e.g. lyophilized powder, which is readily dissolved in sterile solvent immediately prior to use.

In addition to the formulations described previously, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt.

The compositions of the invention and components thereof may comprise soluble polymers as targetable drug carriers.

After pharmaceutical compositions have been prepared, they can be placed in an appropriate container and labeled for treatment of an indicated condition. For administration of a composition of the invention, such labeling would include amount, frequency, and method of administration.

The present invention also includes methods of using the compositions of the invention in combination with one or more additional therapeutic agents including without limitation anti-platelet or platelet inhibitory agents such as aspirin, prioxicam, clopidogrel ticlopidine, or glycoprotein IIb/IIIa receptor antagonists, thrombin inhibitors such as boropeptides, hirudin, or argatroban; or thrombolytic or fibrinolytic agents, such as plasminogen activators (such as tissue plasminogen activator), anistreplase, urokinase, or streptokinase; or combinations thereof.

In addition to being useful in pharmaceutical compositions for the treatment of the cardiovascular conditions described above, one of skill in the art will readily appreciate that the active products, can be used as reagents for elucidating the mechanism of blood coagulation m vitro.

The invention will be described in greater detail by way of specific examples. The following examples are offered for illustrative purposes, and are not intended to limit the invention in any manner.

Those of skill in the art will readily recognize a variety of noncritical parameters which can be changed or modified to yield essentially the same results.

### EXAMPLE 1

Characteristics of several lots of heparin oligosaccharide fractions employed in the present invention and other heparin fractions (UFH from Sigma and enoxaparin from Rhône-Poulenc Rorer) are summarized in Table 2. Listed characteristics include mean molecular weight, activity, anti-IIₐ activity, polydispersity, and K_{d} values. Polydispersity is M_{w}/Mₐ (weight average molecular weight divided by number average molecular weight) and is determined by analyzing data in integration tables for the gel filtration peak profiles of heparin samples. K_{d} values were obtained by measuring the increase in fluorescence (280 nm excitation, 340 nm emission) observed when either AT or IIₐ are titrated with a heparin sample, fitting the titration curve of I/Iₒ verses heparin concentration to a binding isotherm equation, and solving for a (maximum fluorescence change), K_{d} (dissociation constant), and n (moles of heparin required to bind one mole ligand). Bound ligand stoichiometry (1/n) is obtained and interpreted as the proportion of pentasaccharide-containing chains within each heparin fraction.

The selected heparin oligosaccharide fraction, unlike heparin, does not stabilize the binding of thrombin to fibrin. This was demonstrated in studies where addition of fibrin monomer reduced the rate of thrombin inactivation by unfractionated heparin in the presence of both AT and HCII. In contrast, fibrin monomer had only minimal inhibitory effects on the rate of thrombin inhibition by AT or HCII in the presence of equimolar concentrations of the heparin oligosaccharide fraction. Further support that the selected heparin oligosaccharide fraction does not augment thrombin binding to fibrin was obtained by measuring the amount of I¹²⁵ labeled thrombin binding to fibrin clots in the presence or absence of either the heparin oligosaccharide fraction or unfractionated heparin in concentrations ranging from 0 to 7,500 nM.

### EXAMPLE 2

Antithrombotic Activity of Heparin Fractions and Dermatan Sulfate Oligosaccharide Fractions.

An extracorporeal circuit was used to compare the antithrombotic activity of heparin oligosaccharide fractions, dermatan sulfate oligosaccharide fractions, and a combination of the heparin and dermatan oligosaccharide fractions. This circuit is described in detail in Weitz et al. 1999. Briefly, different concentrations of the compound(s) to be tested are added to recalcified human whole blood spiked with ¹²⁵I-labeled human fibrinogen and maintained at 37° C. in a water bath. A peristaltic pump circulates the blood through a 40μ blood filter. Blood clotting within the filter is detected by measuring pressure proximal to the filter with an in-line pressure gauge. Serial blood samples were removed from an in-line reservoir and are counted for residual radioactivity as an index of fibrinogen consumption and clot formation. In addition, starting activated clotting times are measured.

Two key criteria, patency and the percentage of fibrinogen consumed in the clotting process, are used to assess efficacy of compounds tested in this model. Patency is measured as the time to filter failure. The experiment is stopped at 90 minutes, thus defining the maximum time for patency.

The dermatan sulfate oligosaccharide fraction used in the investigation (also referred to in the Examples and Table 3 as LMWDS) has the following characteristics: Mp: 5000 Da, Mw: 7600 Da, and polydispersity of 1.4. The heparinase derived heparin oligosaccharide fraction was obtained by heparinase depolymerization as described herein, and had a peak molecular weight of 8,000, anti-IIa activity of about 100 IU/mg, anti-Xa activity of about 134 IU/ng, and a polydispersity of about 1.5. The nitrous oxide derived heparin oligosaccharide fraction was obtained by nitrous oxide depolymerization as described in PCT/CA98/00548, and it had a molecular weight of 7,700 Da, anti-IIa activity of 84 IU/mg, anti-Xa activity of 123 IU/mg, and a polydispersity of 1.3. The periodate derived heparin oligosaccharide fraction was obtained by periodate depolymerization as described in PCT/EP98/03007 (WO98/55514 published Dec. 10, 1998) and it had a peak molecular weight of 7,900 Da, anti-IIa activity of 19 IU/mg, anti-Xa activity of 43 IU/mg, and a polydispersity of 1.5.

Heparin fractions with a peak molecular weight of about 8,000 Daltons were chosen because this corresponds to chains comprised of about 27 saccharide units. A minimum length of 18 saccharide units is needed to bridge thrombin to antithrombin. Because almost all the chains in these fractions consist of more than 18 saccharide units, they are of sufficient length to catalyze the inactivation of thrombin by antithrombin. In contrast these chains are too short to bridge thrombin to fibrin because this bridging reaction requires chains comprised of 40 saccharide units or more (i.e., 12,000 Da or greater). Consequently, heparin fractions with a mean molecular weight of 8,000 have good inhibitory activity against thrombin by virtue of their ability to activate antithrombin, and are capable of inactivating thrombin bound to fibrin because they do not bridge thrombin to fibrin and render it resistant to inactivation by heparin/antithrombin or heparin/HCII complexes. In contrast, enoxaparin, a commercial low-molecular-weight heparin with a mean molecular weight of about 5,000 (Rhone-Poulenc Rorer, Montreal PQ), is comprised of chains that are mostly too short to bridge thrombin to antithrombin, thereby explaining why its inhibitory activity against thrombin is lower than that against factor Xa.

As previously described (Weitz J I et al. Circulation 1999;99:682-689), different concentrations of each of the fractions was added to recalcified human whole blood spiked with ¹²⁵I-labeled human fibrinogen and maintained at 37° C. in a water bath. A peristaltic pump was then used to circulate the blood through a 40μ blood filter. Clotting of blood within the filter was detected by (a) measuring pressure proximal to the filter with an in-line pressure gauge, and (b) removing serial blood samples from the reservoir and counting residual radioactivity as an index of fibrinogen consumption. Staring activated clotting times were also measured.

As illustrated in Table 3, when used alone, a dermatan sulfate oligosaccharide fraction concentration of 250 μg/ml was needed to maintain filter patency and reduce fibrinogen consumption to <10%. This is noteworthy because in previous work, it was reported that unfractionated dermatan sulf was ineffective in this circuit even at a concentration of 4 mg/ml (Weitz, et al., Circulation, 1999).

Combinations of low molecular weight dermatan sulfate and the 8,000 Da heparinase-derived heparin fraction or enoxaparin were also evaluated LMWDS was effective at 100 μg/ml when combined with 1 μg/ml of the 8,000 Da heparinase-derived heparin fraction, or at 50 μg/ml when combined with 2 μg/ml of this heparin fraction. In contrast, 50 μg/ml LMWDS was ineffective when combined with 3 μg/ml of enoxaparin, with filter failure occurring at 80 min and fibrinogen consumption at 85%. These results indicate that the heparinase-derived fraction is more effective than enoxaparin.

When LMWDS and the 8,000 Da heparinase-derived heparin fractions are used in combination, the two drugs are effective at 5- and 10-fold lower doses, respectively, than those needed to maintain patency when the drugs are used alone (i.e., 50 versus 250 μg/ml of LMWDS and 1 versus 10 μg/ml of the 8,000 Da heparinase-derived fraction).

The combination of LMWDS (50 μg/ml), and heparinase, nitrous acid, and periodate derived heparin fractions, and enoxaparin were compared (all at 2 μg/ml). The combinations of LMWDS and heparinase or nitrous acid-derived heparin fractions were effective. The other two combinations were not effective. (See Table 4).

To further evaluate the heparinase-derived fraction and enoxaparin, the two were compared at gravimetrically equivalent doses (Table 5). Even at 30 μg/ml, enoxaparin was less effective than 10 μg/ml of the heparinase-derived fractions

### EXAMPLE 3

Comparison of the Effect of Fibrin Monomer on the Heparin- and DS-Catalyzed Rates of Thrombin Inhibition by HCII

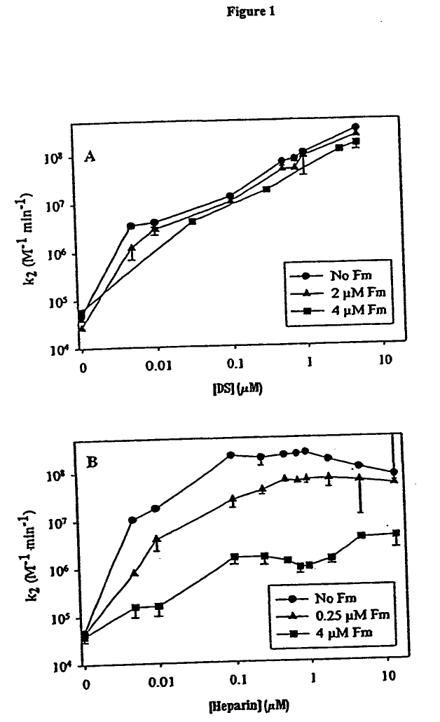

The effect of soluble fibrin monomer, prepared as previously described (15), on the rates of thrombin inhibition by HCII was first examined in the absence or presence of increasing concentrations of DS or heparin. The second-order rate constants (k₂) for inhibition of thrombin by HCII were determined under pseudo-first-order conditions in the absence or presence of 3.3 μM heparin or DS, or 4 μM SF, or both. Thrombin (10 nM) was incubated for 5 minutes at room temperature in TBS containing 0.6% PEG-8000 in the presence of various concentrations of heparin or DS (0 to 11 μM), SF (0 to 4 μM), 10 mM GPRP-NH₂, and 15 mM Tris-HCl, pH 7.5. Reaction mixtures (10 μl) were aliquoted to 96-well round bottom microtitre plates and an equal volume of HCII (in a concentration at least 10-fold higher than that of thrombin) was added to each well at time intervals ranging from 2 sec to 5 min. All reactions were terminated by the addition of 200 μM chromogenic substrate (tGPR-pNA) in 200 μl TBS containing 10 mg/ml polybrene. Residual thrombin activity was calculated by measuring absorbance at 405 nm for 5 min using a Spectra Max 340 Microplate Reader (Molecular Devices, Menlo Park, Calif.). The pseudo-first-order rate constants (k₁) for thrombin inhibition were determined by fitting the data to the equation k₁. t=In([P]ₒ/[P]ₜ), where [P]ₒ is initial thrombin activity and [P]ₜ is thrombin activity at time t. The second-order rate constant, k₂, was then determined by dividing k₁ by the HCII concentration (15). As shown in FIG. 1 (panel A), soluble fibrin (Fm), at concentrations of 2 or 4 μM, causes only a modest 3-fold decrease in the DS-catalyzed rates of thrombin inhibition by HCII. In contrast, Fm causes a dose-dependent decrease in the heparin-catalyzed rates of thrombin inhibition by HCII (panel B). At 1 μm heparin and 4 μM Fm, a maximal 240-fold decrease in the rate was observed, a value consistent with that reported previously (15).

Comparison of the Effect of Fibrin Monomer on the Heparin- and Heparinase-Derived Fraction on the Rates of Thrombin Inactivation by Antithrombin

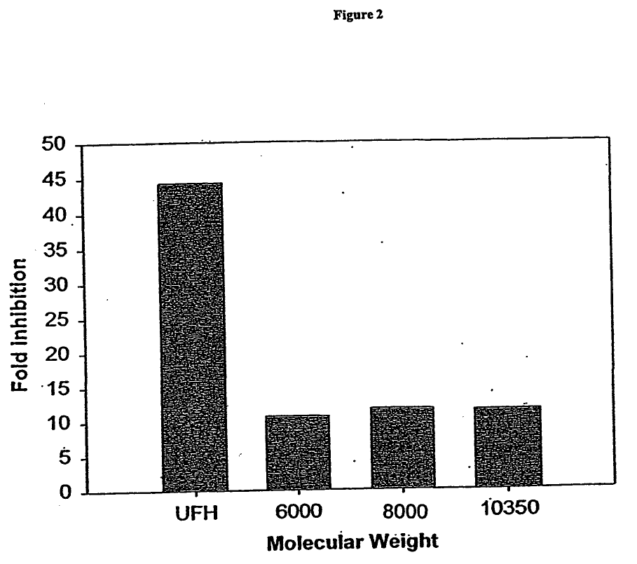

Using similar methodology, the effect of soluble fibrin monomer on the rates of thrombin inactivation by antithrombin in the presence of heparin or the 8,000 Da heparinase-derived fraction were compared. As illustrated in FIG. 2, with heparin, the rate of thrombin inactivation is decreased about 45-fold in the presence of 4 μM fibrin monomer. In contrast, fibrin monomer produces only a 10-fold decreas on the rate of thrombin inactivation with the 8,000 Da heparinase-derived fraction.

Effect of Heparin, DS, or the 8,000 Heparinase-Derived Fraction on the Binding of ¹²⁵I-FPR-Thrombin to Fibrin

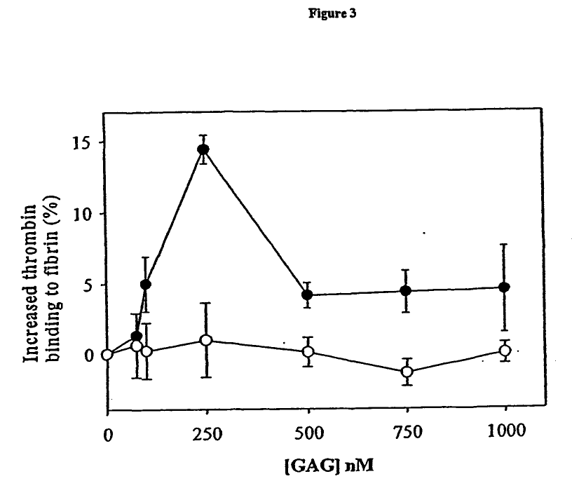

It has been shown previously that heparin enhances the binding of thrombin to fibrin, an effect that occurs regardless of whether heparin has high or low affinity for AT, but occurring only with heparin chains of 11,200 Da or more (18). In this study, the ability of DS to promote thrombin binding to fibrin was compared with that of heparin. To accomplish this, active site-blocked thrombin (FPR-thrombin) and ¹²⁵I-FPR-thrombin were prepared as described (19). The binding of ¹²⁵I-FPR-thrombin to fibrin clots in the absence or presence of either heparin or DS was studied in TBS containing 0.6% PEG-8000 and 0.01% Tween-20. Fibrinogen (7.5 μM was incubated with increasing concentrations of either heparin or DS (0 to 2.5 μM) in a total volume of 40 μl in a series of microsedimentation tubes (catalogue number 72.702, Sarstedt Inc., St. Laurent, PQ). Clotting was initiated by addition of 10 μl of stock A containing 10 mM CaCl₂, 500 nM ¹²⁵I-FPR-thrombin, and 10 nM thrombin. After 45 min incubation at room temperature, fibrin was pelleted by centrifugation for S min at 15,000×g, and aliquots of supernatant were removed for gamma counting. The fraction of thrombin bound to fibrin was calculated as the change in ¹²⁵I-FPR-thrombin binding compared with controls lacking glycosaminoglycans. As shown in FIG. 3, DS has no effect on ¹²⁵I-FPR-thrombin binding to fibrin clots, even at concentrations up to 1 μM. In contrast, at concentrations up to 250 nM, heparin enhances ¹²⁵I-FPR-thrombin binding to fibrin clots in a dose-dependent manner. At heparin concentrations above 250 nM, ¹²⁵-FPR-thrombin binding to clots decreases, likely reflecting the accumulation of distinct heparin-fibrin and heparin-thrombin populations. When thrombin and Fm-Sepharose were titrated with low molecular weight (LMW) heparin (Enoxaparin), there was only a small increase in the amount of thrombin bound, comparable to that observed with DS. The findings with enoxaparin are not unexpected because the heparin chains are too short to bridge thrombin to fibrin.

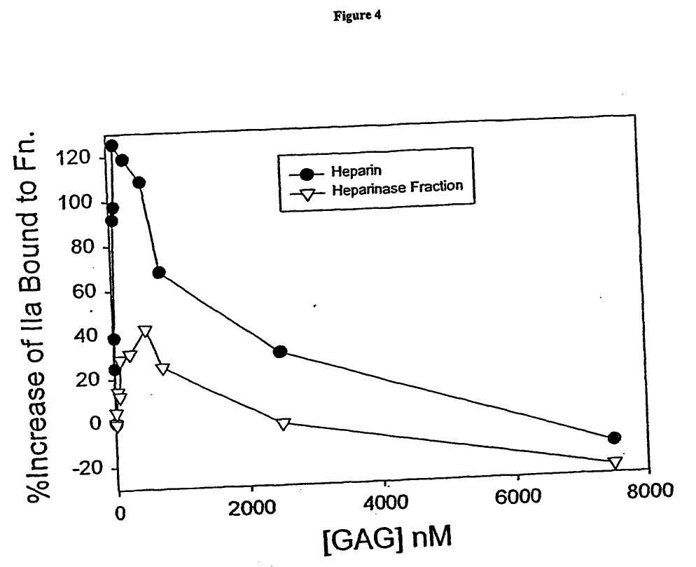

The ability of the 8,000 Da heparinase fraction to promote thrombin binding to fibrin was also compared with that of heparin. As indicated in FIG. 4, the heparinase-derived fraction produced only a small increase in thrombin binding, whereas heparin caused a much greater increase. These findings indicate that lie enoxaparin, the chains within the heparinase-derived fraction also are too short to bridge thrombin to fibrin.

Displacement of IIa from Fm-Sepharose by HCII

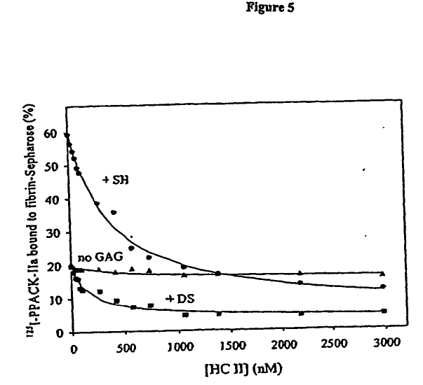

Since catalysis of thrombin inhibition by HCII is not impaired by fibrin in the presence of DS, exosite I on thrombin must be accessible to the DS-HCII complex even though thrombin binds to fibrin via this site. This observation predicts that the DS-HCII complex should be capable of displacing thrombin from fibrin. This was tested by monitoring the amount of ¹²⁵I-FPR-thrombin displaced from Fm-Sepharose by increasing concentrations of HCII in the absence or presence of DS (FIG. 5). HCII alone, at concentrations up to three times physiological, had limited capacity to displace thrombin from Fm-Sepharose. When DS was present at 2.5 μM, dose-dependent displacement of thrombin was obs rved. Maximal displacement was achieved with physiological concentrations of HCII, with half-maximal effect at about 250 nM HCII. Thus, in the presence of DS, the amino-terminus of HCII is able to compete effectively with fibrin for binding to thrombin exosite I. These findings indicate that the DS/HCII complex can displace thrombin from fibrin thereby rendering it susceptible to inactivation.

### EXAMPLE 4

Efficacy Versus Bleeding Studies Rabbit Arterial Thrombosis Prevention Model

A rabbit arterial thrombosis prevention model (Green at al, J. Lab Clin Med. 127:583-587, 1996; Klement et al, 1998 J. Lab Clin Med. 132:181-185, 1998; Klement et al., Blood. 94:2735-2743, 1999) was used to test the efficacy and safety of fractions and compositions of the invention. In the model a rabbit is injected with a test anticoagulant and a small amount of ¹²⁵I fibrinogen. Control animals are given saline in place of anticoagulant Five minutes later, the distal aorta is subjected to balloon endothelial denudation, a stenosis (ligature constriction) is applied to reduce blood flow, and the aortic wall is subjected to an external crush injury from 16 clamps. In the absence of an anticoagulant, the combination of traumatic vessel wall injury and reduced blood flow causes rapid clotting. The extent of clotting can be monitored continuously by measuring blood flood using an ultrasonic flow probe placed distal to the stenosis. The experiment is followed for a total of 90 minutes after injection of the anticoagulant The major efficacy endpoint is the percentage of time that the vessel remains patent over the total 90 minute observation. Safety of various antithrombotic agents can be determined in the same animals using a bleeding ear model which involves making five full-thickness cuts through the rabbit ear and measuring cumulative blood loss over a 30 minute observation period. This represents a merger of two animal models (the arterial thrombosis prevention model with the bleeding-ear model) in order to reduce the number of animals required by 50%. This model designed to study arterial thrombosis prevention, mimics clinical conditions such as unstable angina or clotting after carotid endarterectomy

Heparin Oligosaccharide Fraction

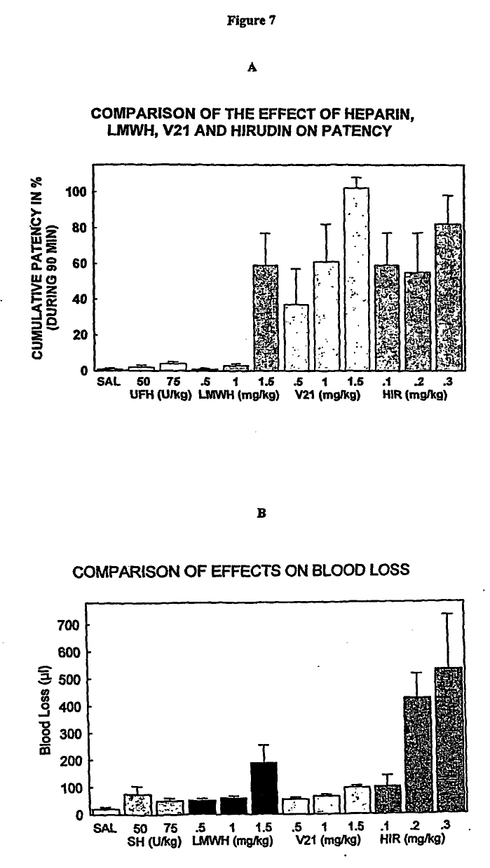

The efficacy and safety of a heparin oligosaccharide fraction (referred to herein as “V21”) was compared with unfractionated heparin, LMWH, hirudin, or saline control in a rabbit arterial thrombosis prevention model. Test compounds were administered 5 minutes prior to creating the arterial stenosis and damage. Blood flow (expressed as % patency) over 90 minutes was measured for efficacy, while the rabbit ear bleeding model was used to measure safety. Rapid clotting was observed in the absence of an anticoagulant (SAL) and at high doses of heparin (UFH). V21 and hirudin (HIR) were much more effective than LMWH at maintaining patency. As shown in FIG. 7, V21 produced 100% patency at doses associated with minimal bleeding (bottom panel), while hirudin demonstrated a much greater propensity for bleeding at the doses required for its efficacy.

Dermatan Sulfate Oligosaccharide Fraction

The efficacy of a dermatan sulfate oligosaccharide fraction (referred to herein as “H2403”) was tested in the rabbit arterial thrombosis prevention and ear bleeding model. A total of 20 rabbits were assigned to four Groups. Rabbits were dosed with concentrations of H2403 from 1 mg/kg to 10 mg/kg as specified in Table 6.

H2403 produced a dose-dependent increase in patency. Patency was between 76-96% for all B2403 doses between 2.5-10 mg/kg, but was only 14% at a dose of 1 mg/kg. The mean time to first occlusion was between 44-54 minutes for H2403-123 doses between 2.5-10 mg/kg, but only 1 minute for the 1 mg/kg dose.

Consistent with these findings, integrated blood flow in the aorta was between 160-288 ml/hr for H2403 doses between 2.5-10 mg/mL but less than 20 ml/hr for 1 mg/kg dose. The range and pattern of blood flow varied considerably from one rabbit to the next, accounting for large standard deviations. The trend toward lower aortic blood flow with lower H2403-123 dose, however, can easily be seen in families of individual rabbit traces as well as in the averaged traces. This data supports the general conclusion that patency occurs at a H2403 dose of 2.5 mg/kg and higher.

A small increase in clot+vessel wall radioactivity (from 15% of reference value to 19%) was seen when the H2403 dose was decreased from 5 mg/kg to 2.5 mg/kg, but a large increase (to 33% of reference value) was seen when the H2403 dose was decreased to 1 mg/kg. This data also supports the general conclusion that a significant increase in efficacy occurs at H2403 doses of 2.5mg/kg and higher in this model.

Safety of LMWDS determined in the same rabbits using the ear bleeding model. Administration of H2403 produced a dose-dependent increase in blood loss with significantly increased bleeding (80 μl and above) occurring at doses greater than 2.5 mg/kg. Patency and occlusion data both suggest that LMWDS efficacy occurs at doses greater than or equal to 2.5 mg/kg.

In summary, based on patency, occlusion time, integrated blood flow, and radiolabeled fibrinogen deposition, by all parameters measured, H2403 shows efficacy in the rabbit arteral thrombosis model at a doses of 2.5 mg/kg or greater. However, significantly increased bleeding occurred at doses greater than 2.5 mg/kg.

Combination of V21 and H2403-123 (Matrix Study)

Both the safety and efficacy of V21+LMWDS formulations were tested on the rabbit arterial thrombosis and prevention model. 100 New Zealand male rabbits were divided into 20 treatment groups of 5 rabbits each (see Table 7), and administered a bolus iv. dose of one V-21/LMWDS formulation per group (dosage matrix Table 8) followed by a continuous infusion repeat dose over the remaining time of the 90 minute experiment.

Dose response curves for the V21 and LMWDS components were compatible with earlier studies, extending the dose range of those studies downward.

V21 had a better efficacy to safety profile than LMWDS, and at the doses tested, both V21 and LMWDS had shallow dose response curves for bleeding.

Addition of LMWDS to V21 did not significantly increase bleeding.

There were additive or synergistic effects of LMWDS on V21for efficacy, but not for bleeding.

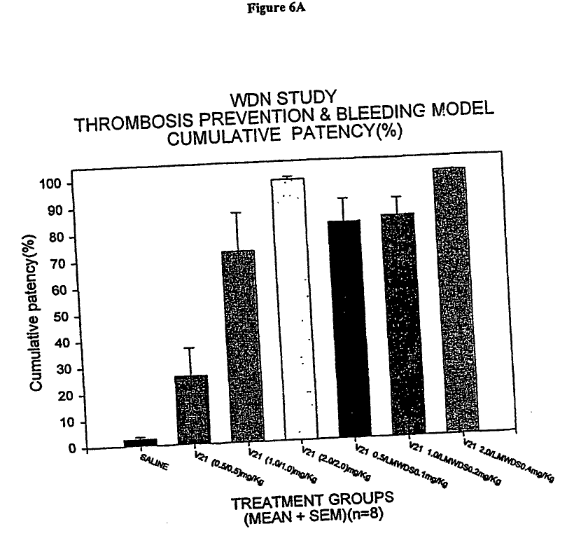

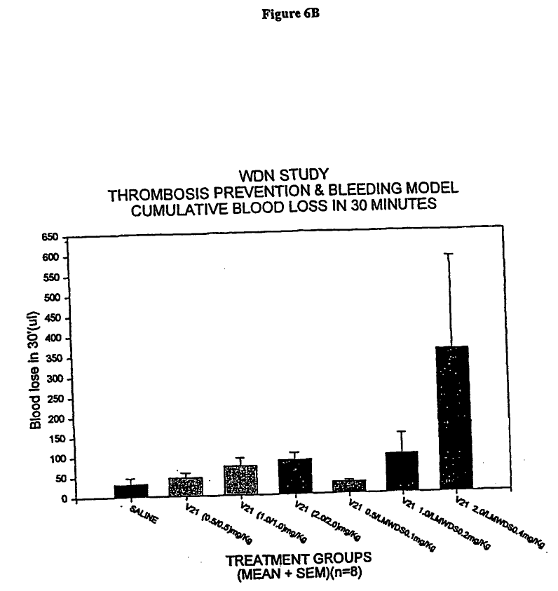

Clot weight, patency, and blood loss were examined simultaneously for V21-LMWDS dose combinations to look for optimal combinations. The following ratios of V21:LMWDS were found to be useful: 1:1, 4:1, and 5:1. Patency and blood loss were examined for the 5:1 combination. Patency was higher in rabbits receiving a combination of V21:LMWDS at a 5:1 ratio compared to rabbits receiving the same dose of V21 alone (FIG. 6A), while bleeding was not increased (FIG. 6B).

### EXAMPLE 5

Baboon Thrombosis Model

A baboon study was undertaken to determine the benefit to risk profiles of V21, LMWDS, and a combination of these agents. The baboon model involved assessment of acute thrombus formation onto a Dacron vascular graft over a period of 60 minutes after placement within an arterio-venous shunt (Hanson et al, Arteriosclerosis 5:595-603, 1985). initially, all animals have a chronic exteriorized silicone rubber shunt surgically placed between the femoral artery and vein. These shunts do not produce measurable platelet activation. To evaluate thrombus formation, a test tubing segment with a Dacron graft (2 cm×4.0 mm i.d.) deployed centrally was inserted into the shunt system and exposed to flowing blood for 1 hour. The shunt tubing segments are standard silicone rubber, 4.0 mm Lid., which is an inherently non-thrombogenic material Blood flow was maintained at 100 ml/min by a clamp placed distal to the test section and was measured continuously using an ultrasonic flowmeter. Autologous baboon platelets were labeled with 1 mCi ¹¹¹-Indium-oxine. Labeling efficiencies averaged >90%. The accumulation of ¹¹¹-In-labeled platelets was measured continuously using a gamma scintillation camera (General Electric 400T). Data were stored at 5 minute intervals and analyzed using a computer-assisted image processing system interfaced with the camera. The total number of deposited platelets was calculated by dividing the deposited platelet radioactivity (counts per minute) by the whole blood ¹¹¹-In-platelet activity (counts per minutes/ml) and multiplying by the circulating platelet count (platelets/ml).

The fibrin content of all thrombi formed after 60 min of blood exposure was measured as follows. Ten minutes before initiating thrombus formation, 5 μCi of ¹²⁵-I-labeled homologous baboon fibrinogen are injected intravenously. After blood exposure for 1 hour, the thrombogenic Dacron graft was thoroughly rinsed with isotonic saline. Due to overlap between the ¹¹¹-In and ¹²⁵-I emission spectra, at least 30 days are allowed for the ¹¹¹-In to decay (half life=2.8 days) before the thrombi are counted for ¹²⁵-I-activity using a gamma counter. Total fibrin accumulation was then calculated by dividing the deposited ¹²⁵-I-radioactivity (counts per minute) by the clottable fibrinogen radioactivity (counts per minute/ml) and multiplying by the circulating fibrinogen concentration (miligrams/ml) as measured in each experiment.

Safety is assessed by direct measurement of bleeding times, as described in Hanson et al Arteriosclerosis 5: 595-603, (1985).

Concurrent control studies were performed in 6 untreated animals. Thrombus formation onto Dacron grafts was assessed as described above, and clotting times (APTT, PT), factor Xa activity (Spectrozyme assay; American Diagnostica), and bleeding times (BT) were measured both immediately pre-graft placement, and at 1 hr following graft placement In addition, 0.5 ml of citrated baboon plasma (for each time point) was removed for determination of thrombin times.

Studies with V21 and LMWDS

V21 was studied first, beginning at total doses of 0.5 mg/kg, 1 mg/kg, and 2 mg/kg (with 50% of the total dose given as a bolus and the remainder given as a continuous infusion over 65 minutes). Five minutes after bolus drug administration, the Dacron graft were placed and platelet imaging performed for an additional 60 minutes, after which, the graft was removed and the study terminated.

LMWDS was studied in an identical fashion, beginning at doses of 2 mg/kg, 5 mg/kg, and 10 mg/kg. At the 2 mg/kg dose 50% of the total dose was given as a bolus and the remainder given as a continuous infusion over 65 minutes. The relative ratio of bolus to infused LMWDS was subsequently adjusted to achieve steady-state clotting times. Thus, at the 5 mg/kg dose the ratios of bolus/infusion amounts were 50%/50% and 33%/67% in two baboons. At the 10 mg/kg dose the ratios were 20-25% bolus to 75-80% inftusion which resulted in constant APTR values over the 60 min insion period.

Subsequently, the administration of V21 and LMWDS was combined in doses of 0.5 mg/kgV21+0.5 mg/kg LMWDS; 1 mg/kg V21+1 mg/kg LMWDS; 2 mg/kg V21+5 mg/kg LMWDS; and 2 mg/kg V21+10 mg/lcg LMWDS (with 50% of the V21 and 20% of the LMWDS doses given as bolus injections and the remaining amounts given as continuous infusions over 65 minutes. This dosing regimen produced nearly constant APTR values during the 60 min infusions. The platelet thrombus imaging, bleeding times, and laboratory measurements were also performed as descrbed herein.

Results of the measurements of platelet thrombus formation after 60 min of blood exposure (or at any earlier time point) were compared using the Student t-test (two-tailed).

Results:

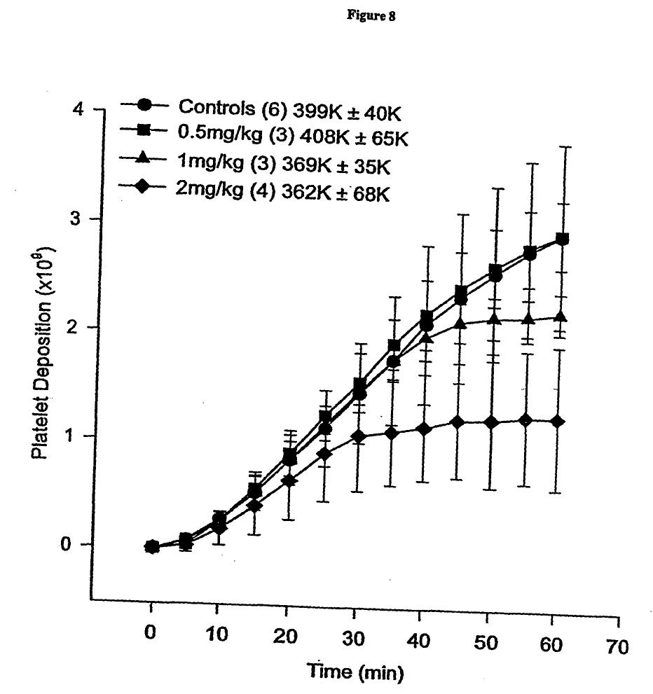

The effects of V21 on Dacron graft thrombosis are shown in FIG. 8. The lower dose of V21, 0.5 mg/kg did not reduce platelet deposition below control values which averaged 2.96±0.85×109 plats after 60 min of blood exposure. The intermediate dose of 1 mg/kg reduced platelet deposition at 60 min by 24% while the higher dose of 2 mg/kg reduced platelet depositin by 57% (FIG. 8).

Fibrin was reduced in a dose dependent manner by V21. At the highest dose, total fibrin accumulation was reduced by 59% (from 2.54±0.37 mg in the controls, versus 1.05±52 mg in the 2 mg/kg treated group). V21 also prolonged the APTT in a dose dependent manner to >200 sec at the 2 mg/kg dose. Measurements of PT were largely unaffected. Bleeding times were not significantly increased by V21, averaging only 5.1 min in the group studies at 2 mg/kg. It may be noted that one animal in the 0.5 mg/kg group recorded a bleeding time of 12 min; however, in light of other observations in this and the other dosing groups the result was viewed as spurious.

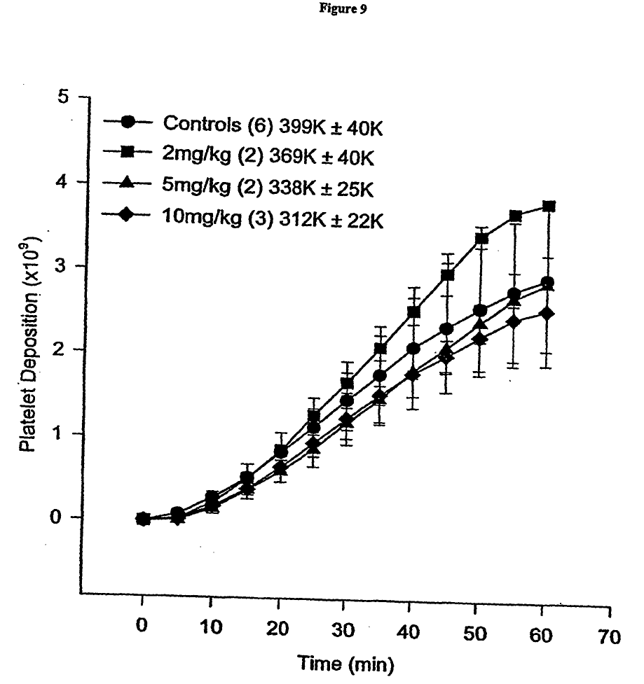

The effects of LMWDS on Dacron graft thrombus formation are given in FIG. 9. Over the range of 2-10 mg/kg, there was no reduction in platelet deposition. Similarly, LMWDS did not reduce fibrin formation on the Dacron graft or prolong the bleeding time. APTT clotting times were prolonged by LMWDS, approximately doubling at the 10 mg/kg dose (mean: 59.1 sec at 60 min. vs. 28.7 sec Pre) PT clotting times were unaffected.

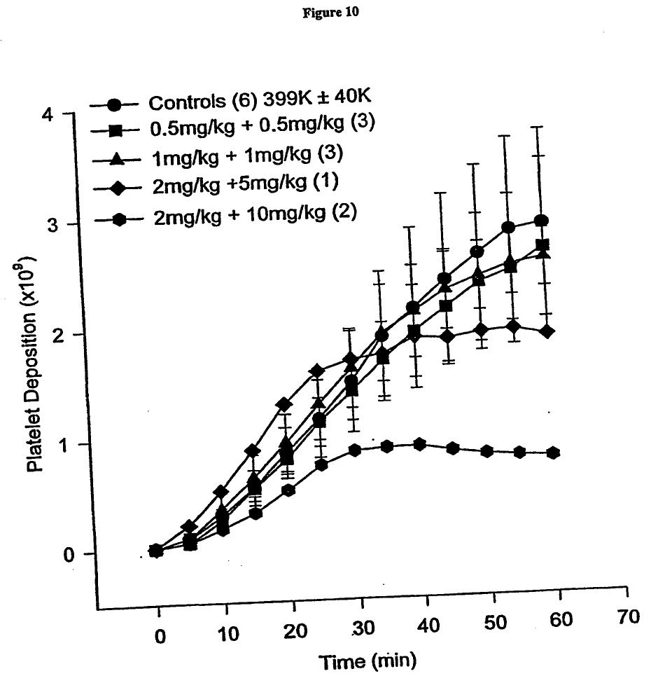

The results for Dacron graft thrombus formation of combining V21 and LMWDS are given in FIG. 10. In combination with 2 mg/kg V21, two baboons were infused with 10 mg/kg LMWDS and one additional animal received 5 mg/kg LMWDS. A significant reduction in platelet deposition was seen at the dose of 2 mg/kg V21+(5-10 mg/kg) LMWDS; however, this effect was comparable to that produced by V21 alone at the same dose (see FIGS. 8 and 10). Similarly, the reduced fibrin deposition seen with the combination therapies was comparable to that produced by V21 alone. Bleeding times were largely unaffected. While PT values were largely unaffected (PT prolonged by 2 sec at the highest doses), the APTT values averaged >500 sec (versus an average of 269 sec for V21 alone at 2 mg/kg).

The baboon model used in these studies is one of rapid flow, which promotes rapid transport and utilization of platelets, combined with a highly thrombogenic surface (Dacron graft) providing a strong initiating stimulus. Under these conditions fibrin formation is not extensive and anticoagulants have generally been ineffective (e.g. standard heparin, pentasaccharide, standard dermatan sulfate). Nonetheless, in this model V21 did reduce arterial platelet and fibrin thrombus formation by >50% without prolonged bleeding times.

TABLE 1

| | Fraction 1 | Fraction 2 | Fraction 3 |
|---|---|---|---|
| Mp (Da) | 8400 | 8300 | 8000 |
| D | 1.3 | 1.6 | 1.5 |
| 0-2 kDa (%) | 0.7 | 3.2 | 2.2 |
| 2-4 kDa (%) | 9.3 | 18.2 | 17.2 |
| 4-6 kDa (%) | 19.0 | 18.5 | 19.2 |
| 6-8 kDa (%) | 20.8 | 15.3 | 15.9 |
| 8-10 kDa (%) | 16.8 | 11.7 | 12.4 |
| 10-12 kDa (%) | 12.5 | 9.3 | 9.4 |
| 12-14 kDa (%) | 8.5 | 7.1 | 7.7 |
| 14-16 kDa (%) | 5.1 | 5.0 | 5.4 |
| >16 kDa (%) | 7.3 | 11.7 | 10.6 |

TABLE 2
Characteristics of Heparin OligoSaccharide
Fraction vs Heparin and LMWH

| | | | Poly- | | | |
|---|---|---|---|---|---|---|
| | | | Dis- | | | |
| | | Mean | per- | K_{d}(AT) | Anti- | K_{d}(IIₐ) |
| Compound | Lot # | MW | sity | nM | IIa | nM |
| Heparin | 200899 | 8,050 | 1.5 | 62 | 100 | 872 |
| Oligosaccharide | 42192-2 | 8,450 | 1.2 | 20 | 67 | — |
| Fraction | 42182-2 | 8,500 | 1.3 | | | |
| | 45262-1 | 8,500 | 1.5 | | | |
| Unfractionated | 36H0763 | ˜15,000 | — | 32 | — | — |
| Heparin | | | | | | |
| (Sigma) | | | | | | |
| Enoxaparin/ | C9005099 | ˜4,500 | — | 47 | — | — |
| LMWH | | | | | | |
| (Rhône- | | | | | | |
| Poulenc) | | | | | | |

TABLE 3
Antithrombotic Activity Of Low Molecular Weight Dermatan
Sulfate (LMWDS) Alone Or In Combination With 8.0 Kda
Heparinase Fraction Or Enoxaparin In An Extracorporeal Circuit

| | | | Time to | | |
|---|---|---|---|---|---|
| | | | Filter | Fibrinogen | Starting |
| LMWDS | Heparinase | Enoxaparin | Failure | Consumption | ACT |
| μg/ml | μg/ml | μg/ml | min | % | sec |
| 250 | — | — | >90 | 9 | 349 |
| 220 | — | — | >90 | 15 | 336 |
| 200 | — | — | 65 | 82 | 342 |
| 100 | 1 | — | >90 | 9 | 320 |
| 80 | 1 | — | >90 | 13 | 316 |
| 60 | 1 | — | 70 | 81 | 273 |
| 50 | 2 | — | >90 | 7 | 308 |
| 50 | 1 | — | 45 | 85 | 296 |
| 25 | 3 | — | 85 | 62 | 339 |
| 25 | 2 | — | 75 | 72 | 288 |
| 50 | — | 3 | 80 | 85 | 294 |

TABLE 4
Antithrombotic Activity Of LMWDS (50 μG/MI) In Combination With
2 μG/MI Of Heparinase, Nitrous Acid Or Periodate Fractions Or
With 2 μG/MI Enoxaparin In An Extracorporeal Circuit

| | Time to | Fibrinogen | Starting |
|---|---|---|---|
| | Filter Failure | Consumption | ACT |
| PREPARATIONS | min | % | sec |
| LMWDS and Heparinase 8,450 | >90 | 11.2 | 292 |
| LMWDS and Nitrous Acid 7,700 | >90 | 16.6 | 299 |
| LMWDS and Periodate 10,100 | 45 | 85.4 | 256 |
| LMWDS and Enoxaparin | 85 | 80.5 | 260 |

TABLE 5
Comparison Of The Antithrombotic Activity Of Enoxaparin And
The Heparinase-Derived Fraction In The Extracorporeal Circuit

| | | Time to Filter | Fibrinogen |
|---|---|---|---|
| | Dose | Failure | Consumption |
| Agent | μg/ml | min | % |
| Enoxaparin | 10 | 25 | 82 |
| | 20 | 70 | 65 |
| | 30 | >90 | 33 |
| Heparinase (8,450 Da) | 10 | >90 | 11 |

TABLE 6
Treatment Groups

| | H2403 | H2403 | |
|---|---|---|---|
| Group | iv bolus | iv infusion | # Animals |
| 1 | 10 mg/kg | 10 mg/kg | 5 |
| 2 | 5 mg/kg | 5 mg/kg | 5 |
| 3 | 2.5 mg/kg | 2.5 mg/kg | 5 |
| 4 | 1 mg/kg | 1 mg/kg | 5 |

TABLE 7
Treatment Groups

| Group | V-21 | LMWDS | # Animals |
|---|---|---|---|
| 1 | Saline | Saline | 5 |
| 2 | 0.125 mg/Kg | — | 5 |
| 3 | 0.25 mg/Kg | — | 5 |
| 4 | 0.50 mg/Kg | — | 5 |
| 5 | 2.50 mg/Kg | — | 5 |
| 6 | — | 0.125 mg/Kg | 5 |
| 7 | — | 0.25 mg/Kg | 5 |
| 8 | — | 0.50 mg/Kg | 5 |
| 9 | 0.125 mg/Kg | 0.125 mg/Kg | 5 |
| 10 | 0.25 mg/Kg | 0.125 mg/Kg | 5 |
| 11 | 0.50 mg/Kg | 0.125 mg/Kg | 5 |
| 12 | 2.50 mg/Kg | 0.125 mg/Kg | 5 |
| 13 | 0.125 mg/Kg | 0.25 mg/Kg | 5 |
| 14 | 0.25 mg/Kg | 0.25 mg/Kg | 5 |
| 15 | 0.50 mg/Kg | 0.25 mg/Kg | 5 |
| 16 | 2.50 mg/Kg | 0.25 mg/Kg | 5 |
| 17 | 0.125 mg/Kg | 0.50 mg/Kg | 5 |
| 18 | 0.25 mg/Kg | 0.50 mg/Kg | 5 |
| 19 | 0.50 mg/Kg | 0.50 mg/Kg | 5 |
| 20 | 2.50 mg/Kg | 0.30 mg/Kg | 5 |

TABLE 8
Dosage Matrix

| V-21/LMWDS | LMWDS (mg/Kg) | | | |
|---|---|---|---|---|
| V-21 | 0.00/0.00 | 0.00/0.125 | 0.00/0.25 | 0.00/0.50 |
| (mg/Kg) | 0.125/0.00 | 0.125/0.125 | 0.125/0.25 | 0.125/0.50 |
| | 0.25/0.00 | 0.25/0.125 | 0.25/0.25 | 0.25/0.50 |
| | 0.50/0.00 | 0.50/0.125 | 0.50/0.25 | 0.50/0.50 |
| | 2.50/0.00 | 2.50/0.125 | 2.50/025 | 2.50/0.50 |

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

It must be noted that as used herein and in the appended claims, the singular forms “a”, “an”, and “the” include plural reference unless the context clearly dictates otherwise.

References Cited in Specification

1. Hish, J. N. Eng. J. Med. 1991; 324:1565-1574.

2. Popma J J, et al., Chest 1995; 108:486-501.

3. Colins R, et al., BMJ 1996; 313:652-659.

4. Hogg P J , Jackson C M, Proc. Natl. Acad. Sci. USA 1989; 86:3619-3623.

5. Weitz J L, et al., J. Clin. Invest. 1990; 86:385-391.

6. Carrie D, et al., Thromb. Haemost 1992; 68:637-641.

7. Matthiason S E, et al., Haemostasis 1995; 25:203-211.

8. Cadroy Y, et al., Arteriosclerosis and Thrombosis 1993; 13:1213-1217.

9. Fernandez F, et al., Br. J Haematol. 1986; 64:309-317.

10. Cohen A T, et al., Thromb. Haemost 1994; 72:793-798.

11. Agnelli G, et al., Thromb. Haemost 1992; 67: 203-208.

12. Prandoni P, et al., Br. J. Sur. 1992; 79:505-509.

13. DiCarlo V, et al., Thromb. Haemost. 1999; 82:30-34.

14. Tollefsen D M, et al., J. Biol. Chem. 1983; 258:6713-6716.

15. Becker D, et al., J. Biol Chem. 1999: 274: 6226-6233.

16. Bendayan P, et al., Thromb. Haemost 1994; 71:576-580.

17. Cosmi B, et al., Thromb Haemost. 1993; 70: 443-447.

18. Hogg P I, Jackson C M, J Biol Chem. 1990; 265:241-247.

19. Fredenburgh J C, et al., J. Biol. Chem. 1997: 272:25493-25499.

We claim

1. A composition comprising a combination of (a) at least one heparin oligosaccharide fraction and (b) at least one dermatan sulfate oligosaccharide fraction for use as a medicament.

2. A composition as claimed in claim 1 wherein the heparin oligosaccharide fraction comprises a mixture of oligosaccharides derived from heparin characterized by one, two, three, four, five, six, or seven or more of the following characteristics:
(a) having antithrombin- and heparin cofactor II (HCII)-related anticoagulant activity in vitro;
(b) the oligosaccharides are too short to bridge thrombin to fibrin, but are of a sufficient length to bridge antithrombin or HCII to thrombin;
(c) having at least 15%, 20%, 25%, 30%, 35%, or 40% oligosaccharides with at least one or more pentasaccharide sequence;
(d) enriched for oligosaccharides having a molecular weight range from about 6,000 to about 12,000, 6,000 to 11,000, or 6,000 to 10,000;
(e) the oligosaccharides have a peak molecular weight of about 7,000 to 10,000, 7,500 to 9,700, or 8,000 to 9,000;
(f) at least 30%, 35%, 40%, 45%, 50%, or 55% of the oligosaccharides have a molecular weight greater than or equal to 6000 Daltons;
(g) at least 200/%, 25%, 30%, 35%, or 40%, have a molecular weight greater than or equal to 8000 Daltons;
(h) apolydispersity of 1.1 to 1.8; and
(i) having similar anti-factor Xa and anti-factor IIa activities.

3. A composition as claimed in claim 2 wherein the heparin oligosaccharide fraction has the characteristics (a), (b), (c) and (cf); (a) (b), (c), and (e); (a), (b), (e), and (f); (a), (b), (e), and (g); (a), (b), (e), (f), (g), (h) and (i); (b), (c), (e), and (g); (b), (d), (c), (h), and (i); (b) (c), (d), and (h); (b), (e), (h), and (i); (b), (e), (f), (h), and (i); (b), (e), (g), (h), and (i); or (a) through (i).

4. A composition as claimed in claim 2 wherein the heparin oligosaccharide fraction has one or more of the following characteristics:
(a) enriched for oligosaccharides with a molecular weight range from 6,000 to 10,000 Daltons, and a mean of about 8,500;
(b) white to off-white crystaline solid;
(c) stable at room temperature; and
(d) a polydispersity of about 1.2 to 1.5.

5. A composition as claimed in claim 2, 3, or 4 wherein the dermatan sulfate oligosaccharide fraction is characterized by one or more of the following:
(a) a sulfur content of 6.0% to 10% (w/w) preferably 6.0 to 8.0% (w/w), more preferably 6.5% to 8% (w/w);
(b) a stlfrte/carboxyl ratio of 1.2 to 2.5, preferably from 1.2 to 2.0, more preferably 1.3 to 1.8, most preferably 1.3-1.6;
(c) a disulfated disaccharide content of 20% to 60% (w/w), preferably 30% to 60% (w/w) of the mono-sulfated disaccharide content;
(d) a heparin cofactor II mediated activity against thrombin in the range 20-60 IU/mg, preferably 30-60 IU/mg.

6. A composition as claimed in claim 2, 3, or 4 wherein the dermatan sulfate oligosaccharide fraction has one or more of the following characteristics:
(a) enriched for oligosaccharides with a molecular weight range from 5,000 to 8,000 Daltons;
(b) white to off-white crystalline solid;
(c) stable at room temperature; and
(d) greater than 6.2% sulfonation by weight

7. A composition as claimed in claim 2, 3, or 4 wherein the dermatan sulfate oligosaccharide fraction comprises a mixture of dermatan polymeric chains with 90% or more having a molecular weight ranging between about 1600 to about 20,000 Daltons and a peak molecular weight of about 4,500 to about 8,000 Daltons.

8. A composition as claimed in any one of claims 1 to 7 wherein the amounts of (a) and (b) are effective to exert a synergistic effect in preventing or inhibiting thrombin generation or activity.

9. A composition as claimed in any one of claims 1 to 8 wherein the doses of the heparin oligosaccharide fraction and dermatan sulfate oligosaccharide fraction are at least 5 to 10 fold lower than the doses of each fraction required to prevent or inhibit thrombin generation or activity in a patient.

10. A pharmaceutical composition comprising a synergistically effective amount of a combination of at least one heparin oligosaccharide fraction and at least one dermatan sulfate oligosaccharide fraction in a pharmaceutically acceptable carrier.

11. A pharmaceutical composition as claimed in claim 10 wherein the pharmaceutically acceptable carrier is adapted to provide the synergistically effective amount of the fractions to inhibit or prevent thrombin generation or activity in a patient.

12. A combination treatment to prevent or inhibit thrombin generation or activity in a patient comprising administering to the patient an effective amount of (a) at least one heparin oligosaccharide fraction; and (b) at least one dermatan sulfate oligosaccharide fraction.

13. A combination treatment as claimed in claim 12 wherein the method provides synergistic activity.

14. A combination treatment as claimed in claim 12 or 13 wherein the heparin oligosaccharide fraction and dermatan sulfate oligosaccharide fraction are administered concurrently or separately.

15. A combination treatment as claimed in claim 12, 13, or 14 wherein the heparin oligosaccharide fraction comprises a mixture of oligosaccharides derived from heparin characterized by one, two, three, four, five, six, or seven or more of the following characteristics:
(a) having antithrombin- and heparin cofactor II (HCII)-related anticoagulant activity in vitro;
(b) the oligosaccharides are too short to bridge thrombin to fibrin, but are of a sufficient length to bridge antithrombin or HCII to thrombin;
(c) having at least 15%, 20%, 25%, 30%, 35%, or 40% oligosaccharides with at least one or more pentasaccharide sequence;
(d) enriched for ollgosaccharides having a molecular weight range from about 6,000 to about 12,000, 6,000 to 11,000, or 6,000 to 10,000;
(e) the oligosaccharides have a peak molecular weight of about 7,000 to 10,000, 7,500 to 9,700, or 8,000 to 9,000;
(f) at least 30%, 35%, 40%, 45%, 50%, or 55% of the oligosaccharides have a molecular weight greater than or equal to 6000 Daltons;
(g) at least 20%, 25%, 30%, 35%, or 40%, have a molecular weight greater than or equal to 8000 Daltons;
(h) a polydispersity of 1.1 to 1.8; and
(i) having similar anti-factor Xa and anti-factor IIa activities.

16. A combination treatment as claimed in claim 15 wherein the heparin oligosaccharide fraction has the characteristics (a), (b), (c) and (d); (a) (b), (c), and (e); (a), (b), (e), and (f); (a), (b), (e), and (g); (a), (b), (e), (f), (g), (h) and (i); (b), (c), (e), and (g); (b), (d), (c), (h), and (i); (b) (c), (d), and (h); (b), (e), (h), and (i); (b), (e), (f), (h), and (i); (b), (e), (g), (h), and (i); or (a) through (i).

17. A combination treatment as claimed in any one of claims 12 to 16 wherein the dermatan sulfate oligosaccharide fraction is characterized by one or more of the following:
(a) a sulfur content of 6.0% to 10% (w/w) preferably 6.0 to 8.0% (w/w), more preferably 6.5% to 8% (w/w);
(b) a sulfate/carboxyl ratio of 1.2 to 2.5, preferably from 1.2 to 2.0, more preferably 1.3 to 1.8, most preferably 1.3-1.6;
(c) a disulfated disaccharide content of 20% to 60% (wtw), preferably 30% to 60% (w/w) of the mono-sulfated disaccharide content;
(d) an activity against thrombin in the range 20-60 IU/mg, preferably 30-60 IU/mg

18. A combination treatment as claimed in any one of claims 12 to 16 wherein the dermatan sulfate oligosaccharide fraction comprises a mnaure of dermatan polymeric chains with 90% or more having a molecular weight ranging between about 1600 to about 20,000 Daltons and a peak molecular weight of about 4,500 to about 8,000 Daltons.

19. A method of inhibiting or preventing thrombin generation or activity in a patient comprising administering in combination, to a patient in need thereof, synergistically effective amounts of at least one heparin oligosaccharide fraction, and at least one dermatan sulfate oligosaccharide fraction.

20. Use of a composition or combination treatment as claimed in any of the preceding claims for preventing, and/or ameliorating disease severity, disease symptoms, and/or periodicity of recurrence of a disease associated with excess thrombin generation or activity.

21. Use of a composition comprising a combination of (a) at least one heparin oligosaccharide fraction and (b) at least one dermatan sulfate oligosaccharide fraction for the preparation of a medicament for the prevention or inhibition of thrombin generation or activity.

22. Use of synergistically effective amounts of at least one heparin oligosaccharide fraction, and at least one dermatan sulfate oligosaccharide fraction in the preparation of a pharmaceutical composition for inhibiting or preventing thrombin generation or activity in a patient

23. A kit form of a composition as claimed in any of the preceding claims.
